(19) United States
(12) Patent Application Publication
(10) Pub. No.: US 2016/0151582 A1
(43) Pub. Date: Jun. 2, 2016

(54) INDICATION ASSEMBLY

(71) Applicant: SANOFI, Paris (FR)

(72) Inventors: Tom OAKLEY, Cambridge Cambridgeshire (GB); Matt SCHUMANN, Bourn, Cambridge, Cambridgeshire (GB); Stuart MILNE, Buckden St. Neots, Cambridgeshire (GB)

(21) Appl. No.: 14/905,454

(22) PCT Filed: Jul. 17, 2014
(86) PCT No.: PCT/EP2014/065336

§ 371 Date: Jan. 15, 2016

(30) Foreign Application Priority Data
Jul. 17, 2013 (EP) 13176879.8

(51) Int. Cl. A61M 5/315

(52) U.S. Cl. CPC A61M 5/31553; A61M 2005/3126

(57) ABSTRACT

An indication assembly (100) is presented. The indication assembly (100) comprises indication members (110, 120) further comprising interaction features (111, 121). The indication assembly (100) comprises a locking member (130) which is movable with respect to the indication members (110, 120) between a locking position and a non-locking position. The locking member (130) comprises a locking member feature (131), wherein the indication members (110, 120) are rotatable relative to the locking member (130) around an axis (x). The indication assembly (100) is configured such that when the locking member (130) is in the non-locking position, the indication members (110, 120) are rotatable by a predetermined angle in a first rotational direction. The indication assembly (100) is configured such that when the locking member (130) is in the non-locking position and the indication members (110, 120) are rotated by the predetermined angle with respect to the locking member (130), the locking member feature (131) interacts with the interaction feature (111) such that the locking member (130) is displaced into the locking position.

The present disclosure relates to an indication assembly, e.g. an indication assembly for a drug delivery device, and to a drug delivery device.

Drug delivery devices are, for example, known from U.S. Pat. No. 5,383,865 A, U.S. Pat. No. 7,699,815 B2 and WO 2006/089734 A1.

It is an object of the present disclosure to provide an assembly which facilitates an indication, e.g. an indication of a dose set with a drug delivery device. Further, a drug delivery device should be provided.

This object is achieved by the subject-matter of the independent claim. Advantageous embodiments and refinements are subject-matter of the dependent claims.

One aspect of the present disclosure relates to an indication assembly comprising an indication member. The indication member comprises an interaction feature, whereby the indication member is rotatable. The indication assembly further comprises a locking member being movable with respect to the indication member between a locking position and a non-locking position. The locking member comprises a locking member feature. The indication member is rotatable relative to the locking member around an axis. The axis may be a longitudinal axis of the indication assembly.

In an embodiment, the indication assembly is configured such that when the locking member is in the non-locking position, the indication member is rotatable by a predetermined angle with respect to the locking member in a first rotational direction. Advantageously, thereby a movement range is provided in which the indication member may effect an indication of information. The indicated information may change when the indication member is rotated.

In an embodiment, the indication assembly is configured such that when the locking member is in the locking position, the indication member is rotationally locked with respect to the locking member in the first rotational direction. With this embodiment, it is advantageously achieved that an indication of information to be indicated by the indication assembly is restricted. Particularly, a limit may be provided therewith up to which information may be indicated. Rotation of the indication member in the rotational direction opposite to the first direction may be allowed when the locking member is in the locking position.

In an embodiment, the indication assembly is configured such that when the locking member is in the non-locking position and the indication member is rotated by the predetermined angle with respect to the locking member, the locking member feature interacts with the interaction feature such that the locking member is displaced into the locking position with respect to the indication member, thereby rotationally locking the indication member with respect to the locking member in the first rotational direction. The indication assembly can advantageously be used, e.g. for an indication of a set dose of a drug delivery device. Particularly, the indication assembly provides the advantage of a low number of interacting parts, as e.g. a first and a second indication member and a locking member, such that it can be safely applied in a drug delivery device. The indication assembly is not restricted to delivery device, especially drug delivery devices. The indication assembly could also be applied in odometers for distance measurements and/or counting devices in general. The predetermined angle may be an angle by which the indication member may be rotated during an operation of the indication assembly. The predetermined angle may relate to the range of information which is to be indicated by the indication assembly. Preferably, the locking member may be connected to, preferably rotationally locked to, a further component of the device in which the indication assembly is applied.

In an embodiment, the indication assembly is configured such that a movement of the locking member between the locking position and the non-locking position is or comprises an axial displacement along the axis. According to this embodiment, the indication member can easily and expediently be locked against the rotation with respect to the locking member in the first rotational direction.

In an embodiment, the indication assembly is configured such that when the locking member is arranged in the locking position, the indication member is rotatable in a second rotational direction with respect to the locking member, wherein the second rotational direction is opposite to the first rotational direction. When the indication assembly is applied in a drug delivery device, a rotation of the indication member in the first rotational direction may relate to a dose setting operation and the rotation of the indication member in a second rotational direction may relate to the operation of decreasing the size of a previously set dose or of cancelling a previously set dose. Advantageously, this embodiment still allows a rotation of the indication member with respect to the locking member in a second rotational direction while the indication member is rotationally locked with respect to the locking member in the first rotational direction. Particularly, a reversible or a resettable functionality may be attained in this way.

In an embodiment, the interaction feature comprises a stop face and a guiding surface. According to this embodiment, a rotational locking between the indication member and the locking member may advantageously established.

In an embodiment, the stop face has a surface normal which is perpendicular to the axis, wherein the guiding surface is arranged adjacent to the stop face and the guiding surface is arranged to guide the locking feature against the stop face such that the indication member is rotationally locked with respect to the locking member in the first rotational direction by abutment of the locking feature and the stop face. In this way, the rotational locking of the indication member is expediently facilitated.

In an embodiment, the locking member is axially guided by a locking member guide. Such a locking guide member may, e.g., be a housing component of the device to which the indication assembly is applied.

In an embodiment, the indication member is a first indication member.

In an embodiment, the interaction feature is a first interaction feature.

In an embodiment, the indication assembly comprises a second indication member. The second indication member is rotatable relative to the locking member around the axis. The second indication member comprises a second interaction feature. Owing to a second indication member, a parametric space of information to be indicated by the indication member can advantageously be increased. Preferably, the first and the second indication member are mechanically decoupled from one another. Particularly, they may be rotated independently from each other.

In an embodiment, the first and the second indication member cooperate to define the information indicated by the indication assembly. Thereby, a multiplicity of relative orientations and/or positions of the first and the second indication member may contribute to the information to be indicated by the indication assembly.

In an embodiment, the indication member comprises indices which are disposed circumferentially around the axis of rotation. The indices may comprise numbers which display information such as the number of units of drug set to be dispensed by a drug delivery device.

In an embodiment, the locking member feature is a first locking member feature and the locking member comprises a second locking member feature. The second locking member feature is arranged and configured such that it rotationally locks the second indication member in the first rotational direction with respect to the locking member when the first and/or the second indication member are rotated by the predetermined angle in the first rotational direction with respect to the locking member. The locking member features may be spaced apart from each other. The respective indication member, particularly an indication surface of this indication member may be arranged between the locking member features. The first and the second indication member may be rotatable by different predetermined angles. Advantageously, the previous embodiment allows for a simultaneous locking of the first and the second indication member during an operation of the indication assembly.

In an embodiment, the guiding surface is arranged obliquely with respect to an axis of rotation. Thereby, it can be expediently achieved that the locking member is displaced into the locking position.

In an embodiment, the second interaction feature comprises a recess and the guiding surface and the recess are configured such that when the first and/or the second indication member are rotated by the predetermined angle in the first rotational direction with respect to the locking member, the locking member is guided by the guiding surface such that the second locking member feature is displaced into the recess. As an advantage of this embodiment, the first and the second indication member may be simultaneously rotationally locked with respect to the locking member in the first rotational direction.

In an embodiment, the first and the second indication member are configured such that in a first position of the first and the second indication member, the first and the second indication member are rotationally locked by and with respect to the locking member in the second rotational direction, and in a second position of the first and the second indication member, the first and the second rotation member are rotatable with respect to the locking member in the second rotational direction. As an advantage thereof, a common initial position of the first and the second indication member may be provided by the first position which may, e.g. correspond to a “zero dose” position, when the indication assembly is applied in a drug delivery device. In the first position of the first and the second indication member, the locking member is preferably also in an initial (axial) position. The second indication member may comprise a stop which—in the mentioned first position—abuts the locking member such that the first and the second indication member are rotationally locked by and with respect to the locking member in the second rotational direction.

In an embodiment, the first indication member comprises a cut-out and the second indication member comprises a stop.

In an embodiment, the indication assembly is configured such that in the initial position the first locking member feature is arranged in the cut-out and the locking member is arranged such that rotation of a first and a second indication member with respect to the locking member in the second rotational direction is prevented by an interaction of the locking member with the stop. Particularly, this embodiment may enable the simultaneous rotational locking of the first and the second indication member. During a rotation of the second indication member in the first rotational direction, the locking member is preferably moved out of the initial position and into the non-locking position. The initial position may be the most proximal position of the locking member with respect to the first and the second indication member.

In an embodiment, the locking member is biased towards the non-locking position by a biasing member. The biasing direction may be the proximal direction. Thereby, a defined initial (axial) position of the locking member may be provided.

The “proximal position” or “proximal direction” of the indication assembly may mean the position or direction which is or directs furthest away from the dispensing end of a drug delivery device the indication assembly is applied in.

The “distal position” or “distal direction” of the indication assembly may mean the position or direction which is or directs closest to the dispensing end of the drug delivery device the indication assembly is applied in.

In an embodiment, the first and the second locking member feature are arranged at or near opposite ends of the locking member and the first and the second indication member are at least partially arranged axially between the first and the second locking member feature. The locking member may partially surround the first and the second indication member. According to this embodiment, the first and the second locking member feature may preferably interact with the first and the second interaction feature which may be arranged at axial side faces of the first and the second indication members, respectively.

A further aspect of the present disclosure relates to a system comprising the indication assembly and a driver. The driver may be provided to drive movement of the indication member such that the indicated information changes.

In an embodiment, the driver is coupled to the first and/or the second indication member. The driver may be coupled to the first and/or the second indication member in order to drive the first and the second indication member during an operation of the indication assembly. Thus, the driver may be used to adjust the relative movement of the first and second indication member such that the desired information is indicated in a specific relative rotational position of the indication members.

In an embodiment, the driver comprises a first pinion and a second pinion and the first indication member comprises a first corresponding pinion being coupled to the first pinion and a second indication member comprises a second corresponding pinion being coupled to the second pinion. The driver is suitable and configured to drive the first and the second indication member via the first and the second corresponding pinion. According to this embodiment, it is expediently achieved that the first and the second indication member are driven or actuated during the operation of the indication assembly.

In an embodiment, the first indication member is incrementally rotatable and the coupling is configured such that when the second indication member is rotated by one revolution, the first indication member is rotated by one increment. According to this embodiment, the indication assembly may expediently be configured as a counting mechanism.

In an embodiment, the first and/or the second indication member comprise indicia such as numbers or symbols which provide or contribute to the information to be indicated by the indication assembly.

In an embodiment, the first and the second indication member are counter wheels.

A further aspect of the present disclosure relates to a drug delivery device comprising the system and a housing having a proximal and a distal end. The longitudinal axis of the indication assembly may extend through the proximal and the distal end of the drug delivery device when the indication assembly is mounted to the drug delivery device.

In an embodiment, the driver of the system may relate to or a dose member of the drug delivery device.

In an embodiment, the drug delivery device is a variable dose device, wherein the size of a dose of drug to be dispensed by the device can be set by a user between a minimum dose and a maximum dose.

In an embodiment, the indication assembly is adapted to indicate the size of the currently set dose. Thereby, it is achieved that the user may view or inspect the size of the currently set dose.

In an embodiment, when the locking member is in the locking position, the maximum dose is set. Thereby, an important safety and information tool is provided, e.g. for a user of the drug delivery device, in that the user is given feedback that the maximum number of doses to be dispensed from the drug delivery device is reached. Moreover, this provides the advantage that the user is prevented from setting an additional dose, when the maximum dose has already been set. A further aspect of the present disclosure relates to the use of the indication assembly or the system as a display mechanism for a drug delivery device, preferably as a dose display mechanism, e.g. a dose display mechanism which simultaneously provides a maximum settable dose stop which defines the maximum dose of drug which can be set to be dispensed by the drug delivery device in a single dispensing action.

Features which are described herein above and below in conjunction with different aspects or embodiments may also apply for other aspects and embodiments. Particularly, features described with respect to the arrangement may apply for the method, the unit and the module and vice versa.

Further features and advantages of the subject matter of this disclosure will become apparent from the following description of the exemplary embodiment in conjunction with the figures, in which:

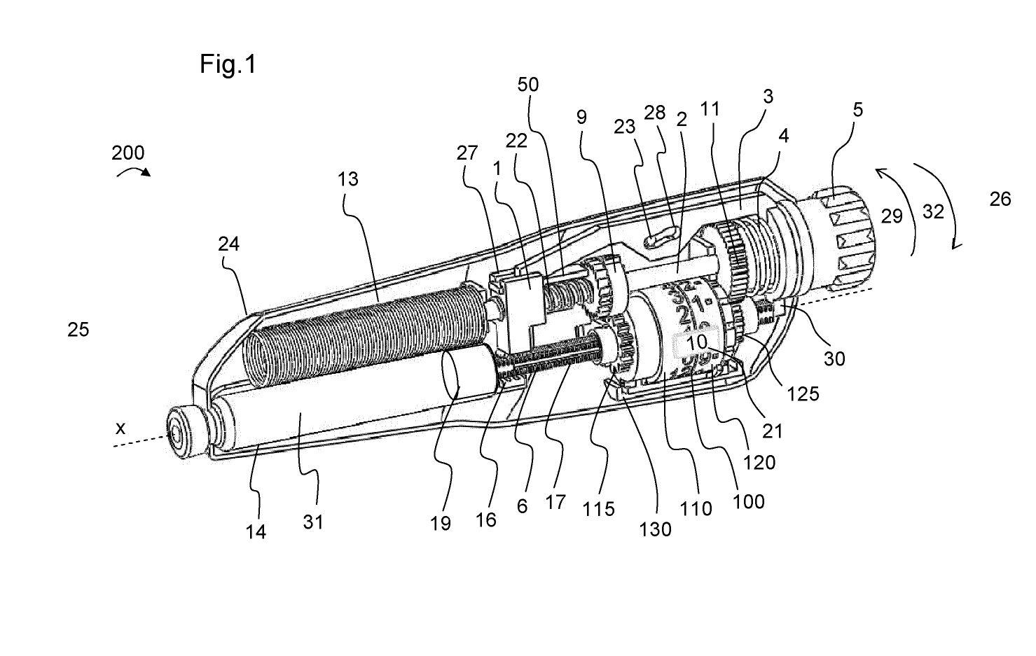

FIG. 1 shows a perspective view of components of a drug delivery device.

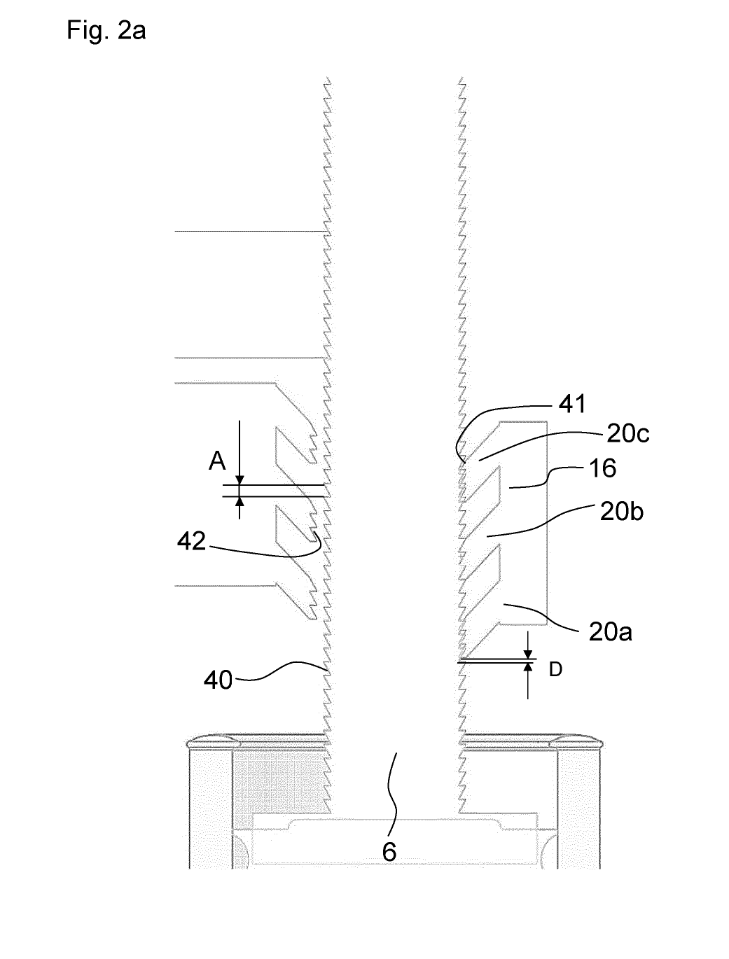

FIGS. 2a to 2c show the schematics of a side view of components of a drive mechanism for a drug delivery device.

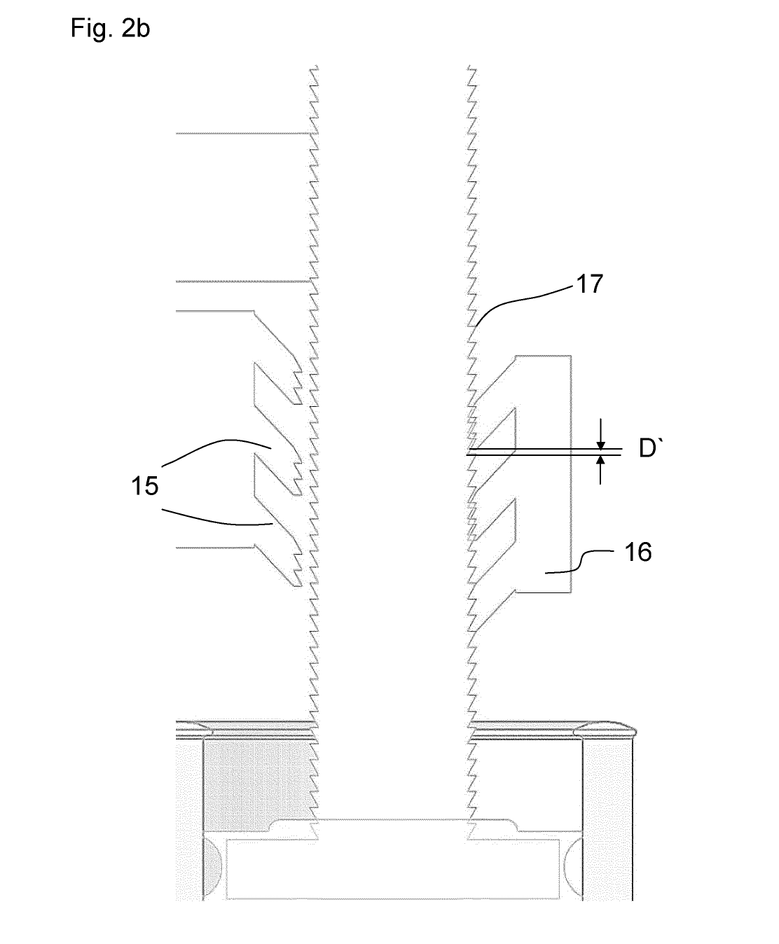

FIGS. 3a and 3b show a partial perspective view of components of the drug delivery device. FIG. 3a shows a situation in which a clutch mechanism is engaged and FIG. 3b shows a situation in which the clutch mechanism is disengaged.

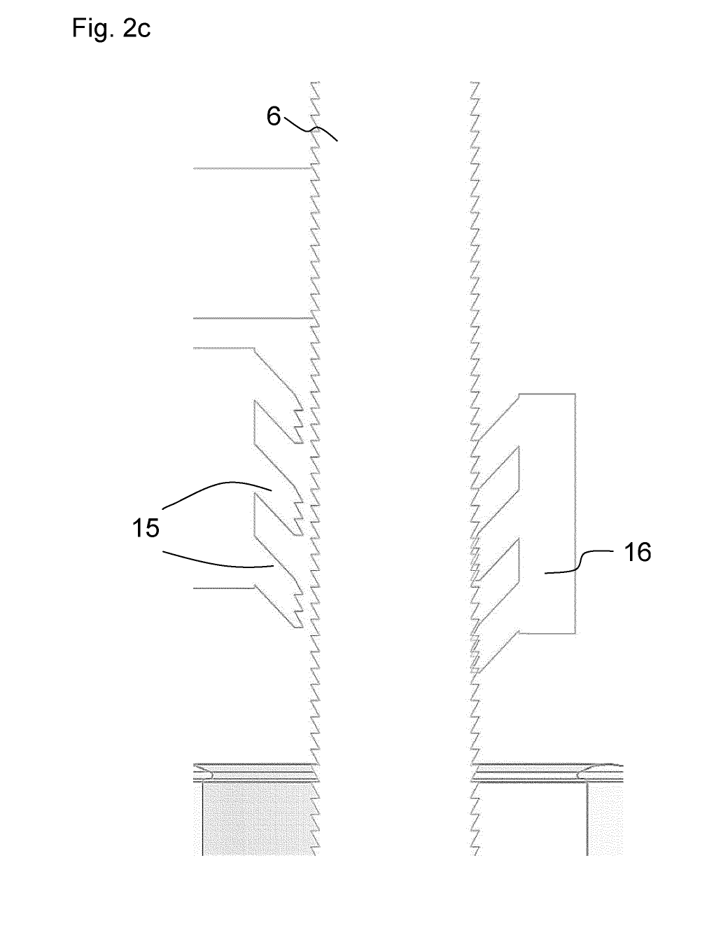

FIG. 4 shows a perspective view of components of the drug delivery device.

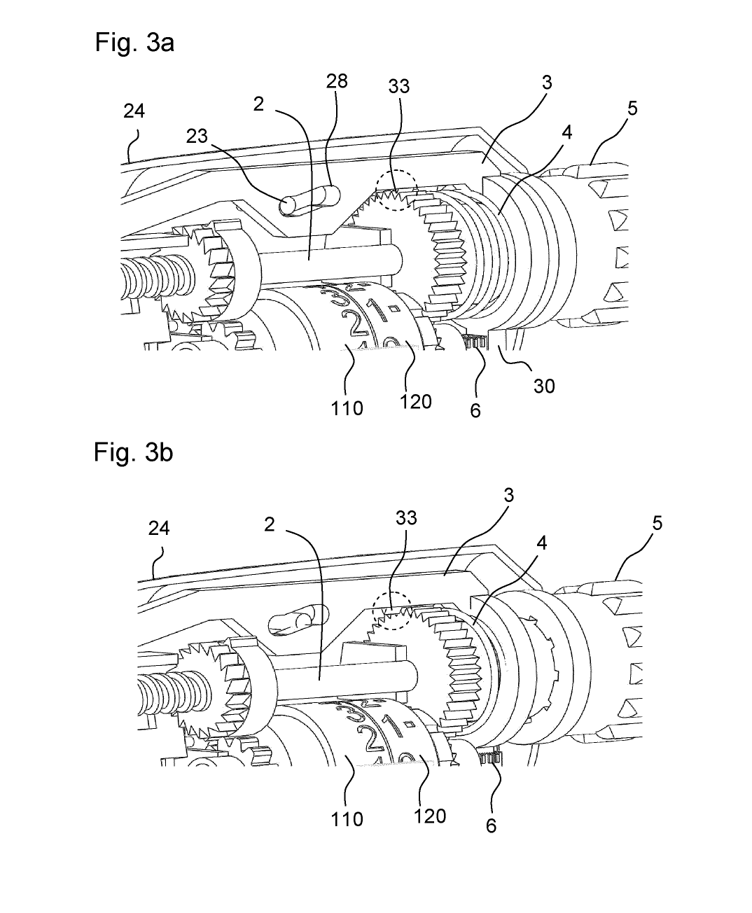

FIG. 5 shows a perspective view of further components of the drug delivery device.

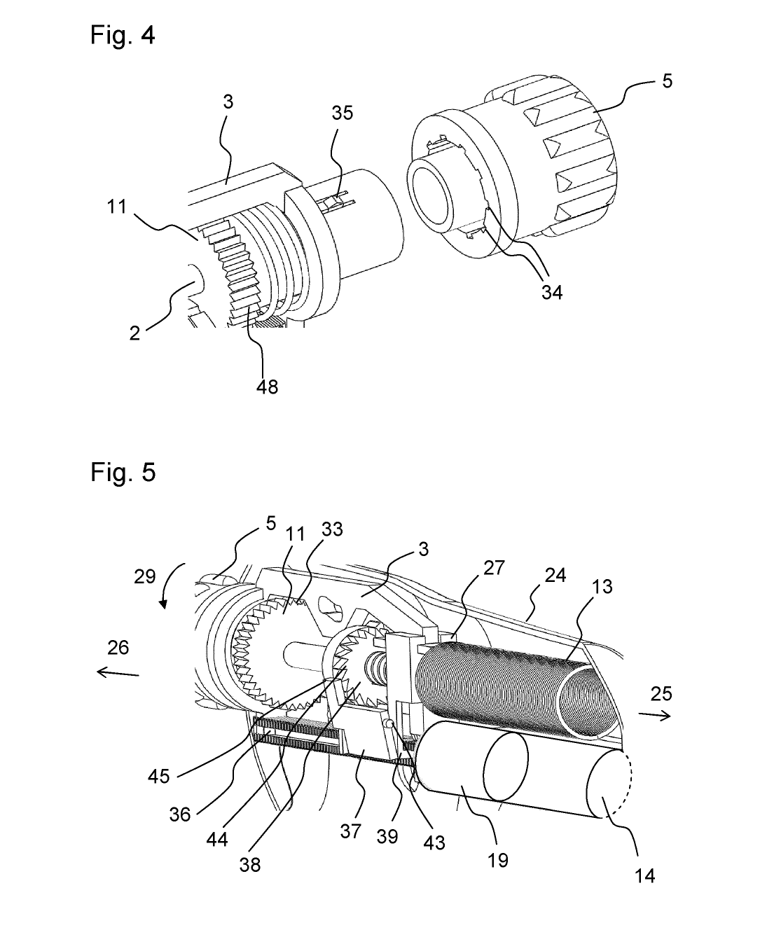

FIG. 6 shows a side view of components of the drug delivery device.

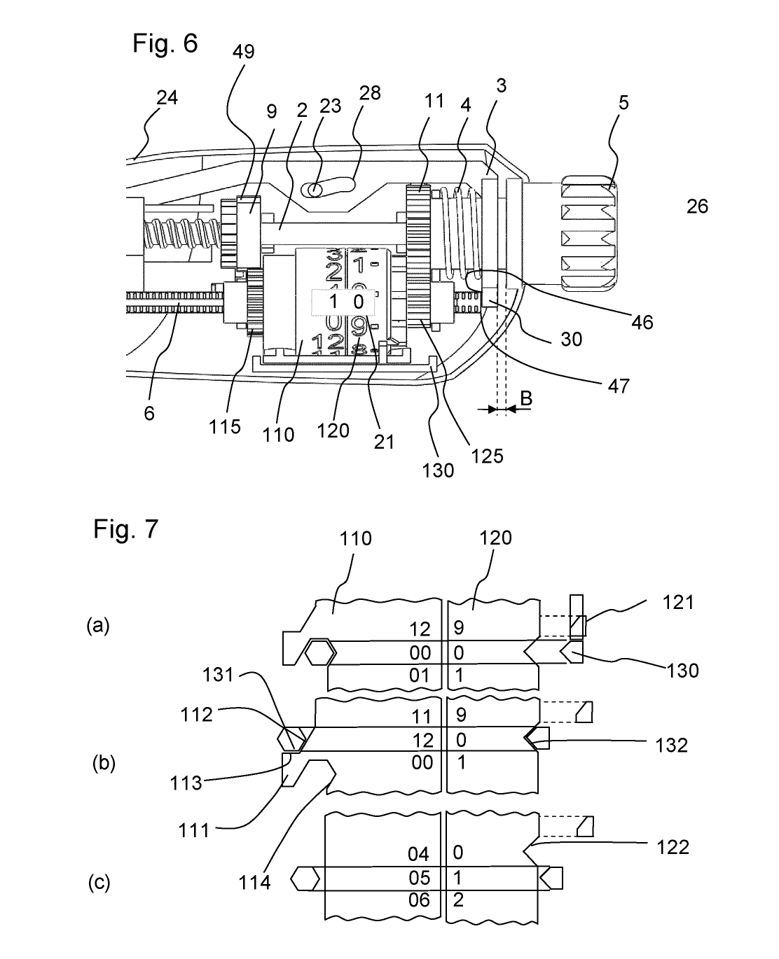

FIGS. 7A to 7C show the schematics of an indication mechanism of the drug delivery device, respectively.

Like elements, elements of the same kind and identically acting elements may be provided with the same reference numerals in the figures. Additionally, the figures may be not true to scale. Rather, certain features may be depicted in an exaggerated fashion for better illustration of important principles.

FIG. 1 shows a perspective view of a drug delivery device 200. The drug delivery device may be a disposable drug delivery device. The drug delivery device 200 comprises a housing 24 which houses further components. Only one half of the housing 24 is shown in FIG. 1 such that inner components of the drug delivery device 200 are visible. The drug delivery device 200 further comprises a drive member 1 and a piston rod 6. The piston rod may have a cross-section resembling a square, rectangle, parallelogram, circle or ellipse.

The drive member 1 is configured to move the piston rod 6 in a distal direction, e.g. during a dose delivery of the drug delivery device 200. The drug delivery device 200 comprises a longitudinal axis x, a distal end 25 and a proximal end 26. Preferably, the longitudinal axis x extends through the distal end 25 and the proximal end 26. In other words, the distal end 25 and the proximal end 26 may be spaced along the longitudinal axis. The drug delivery device 200 further comprises a, preferably replaceable, cartridge 14 in which a plunger 19 is retained. The piston rod 6 may be arranged next to or abut the plunger 19. The cartridge 14 may further contain a drug 31 or medical substance to be dispensed from the drug delivery device 200. The drug 31 may be dispensed in measured doses. The drug 31 may be retained in the cartridge 14. The cartridge 14 may contain 1.5 ml or 3 ml of the drug 31. Preferably, the cartridge 14 is arranged or aligned longitudinally. The piston rod 6 may also be arranged or retained longitudinally such that it is movable with respect to the cartridge 14. The drug delivery device 200 further comprises a dose member 2 which may effect a dose setting and a dose dispensing of the drug delivery device. The dose member 2 is threadedly engaged with the drive member 1, e.g. via an outer thread 22. Accordingly, the drive member 1 may comprise an inner thread matching with the outer thread 22. Thereby, said threaded engagement may be configured such that during a rotation of the dose member 2 with respect to the housing 24, the drive member 1 is axially moved. In a setting mode of operation, the dose member is rotatable in a first or second direction with respect to the housing to set a dose and in a dispensing mode of operation, the dose member 2 is rotatable in a second direction opposite to the first direction with respect to the housing to dispense a set dose. The drug delivery device 200 further comprises a displacement member 3 which may be configured to displace or to contribute to the displacement of the drive member 1.

The dose member 2 and the displacement member 3 are preferably aligned parallel to the longitudinal axis x but arranged radially offset from the cartridge 14 and the piston rod 6. Drive member 1 may at least partially be arranged between the piston rod 6 and the dose member 2. A longitudinal axis of the drive member may thereby be aligned radially. The dose member 2 and the displacement member 3 may comprise an elongate shape, respectively. The displacement member 3 is engaged to the drive member 1 via a guidance 27 of drive member 1. The guidance 27 may be configured such that, e.g., when the dose member 2 is rotated, the drive member 1 is rotationally locked with respect to the displacement member 3 such that the dose member 2 and the drive member 1 are rotated relative to each other. The drug delivery device 200 further comprises a spring element 4 and a dose button 5. The spring element 4 is retained between a pinion 11 of the dose member 2 and the dose button 5. The displacement member 3 comprises a drive member displacement member 50 which comprises an elongate shape and which is aligned parallel to the longitudinal axis x. The length of the drive member displacement member 50 may relate to the travel of the drive member 1 and in this way to the amount of drug 31 to be dispensed during the dispensing of drug from the filled cartridge. The drive member displacement member 50 is retained by the guidance 27. Preferably, the guidance 27 rotationally and radially locks the drive member displacement member 50, whereby only relative axial movement of said components is allowed.

The displacement member 3 comprises a rod displacement feature 30. In the depicted situation, the drug delivery device 200 is in an initial state. During the first use of the drug delivery device 200, the rod displacement feature 30 may abut the piston rod 6. The drug delivery device 200 further comprises an indication assembly 100. The indication assembly 100 comprises a first indication member 110 and a second indication member 120. The piston rod 6 extends through the first and the second indication members 110, 120. The indication assembly 100 further comprises a locking member 130 which is configured to rotationally lock the first and the second indication member 110, 120 with respect to the housing 24. The drug delivery device 200 further comprises a guide feature 23 which is fixed to the housing 24. When, e.g. the dose button 5 is moved distally, the displacement member 3 is also moved distally against the resilience of the spring element 4. Thereby, the displacement member 3 is guided by the guide feature 23 which is engaged to a bore 28 of the displacement member 3. Via the bore 28 and the guide feature 23, an axial movement of the displacement member 3 may at least partly be converted into a radial movement of the displacement member 3 and/or the drive member displacement member 50. The displacement member 3 may comprise a certain flexibility.

The drug delivery device 200 further comprises a drive spring 13 which is configured such that it is loaded upon a rotation of the dose member 2 in a first direction (cf. arrow 29). To this effect, a distal end of the drive spring 13 is preferably fixed to the housing 24 and a proximal end of the drive spring 13 is preferably fixed to, e.g. a distal end of the dose member 2. The drive spring 13 is a torsion spring. During setting of a dose of drug 31, the drive spring 13 is loaded and spring energy is stored which can be used for a delivery of the drug 31. The displacement member 3 is rotationally locked with respect to the housing 24. In the setting mode of operation, the dose button 5 is rotated in the first direction 29. The dose button 5 is connected to the dose member 2, wherein said connection is configured such that the dose member 2 is also rotated in the first direction along with the dose button 5, such that the drive spring 13 is loaded. Due to the threaded engagement of the dose member 2 and the drive member 1, in the setting mode of operation, the drive member 1 is moved proximally while in the dispensing mode of operation, the drive member 1 is moved distally. In the setting mode of operation, the dose button 5 may also be rotated in a second direction (cf. arrow 32) opposite to the first direction 29 in order to decrease or cancel a set dose of drug 31. This would move drive member 1 distally. The dose button may be operated, e.g. rotated to dial the size of a dose to be delivered. The size may span a range between zero units and a maximum number of gradations or units, e.g. 120.

The displacement member 3 and the dose member 2 are coupled via a releasable clutch mechanism by which said components are rotationally lockable. The releasable clutch mechanism is configured to withstand the spring force of the drive spring when a dose is set. When a maximum settable dose is set, the releasable clutch mechanism has to withstand a maximum spring force. The releasable clutch mechanism can be released by distal movement or depressing of the dose button 5 with respect to the housing 24. Thereby, the displacement member 3 and the dose member 2 are disengaged (cf. FIG. 3 further below).

The spring element 4 biases the releasable clutch mechanism towards the engaged state. In the setting mode of operation, the drive member is preferably in a first position, wherein it is disengaged from the piston rod 6. When the dose button 5 is depressed or moved distally which is preferably manually performed by a user of the drug delivery device 200, it is preferably switched from the setting mode to the dispensing mode of operation. During distal movement of the dose button 5 and the displacement member 3 with respect to the housing 24, the displacement member 3 is guided via the guide feature 23 in such a manner that the displacement member 3 displaces the drive member 1 radially, i.e. towards the piston rod 6 such that the piston rod 6 is displaced from the first to a second position. In the second position, the drive member 1 is engaged to the piston rod 6. The drive mechanism is preferably configured such that at the same time or slightly after the drive member 1 has engaged the piston rod, the releasable clutch mechanism is released and the spring force of the loaded drive spring 13 drives the dose member 2 such that the dose member 2 is rotated in the second direction 32. The drive spring 13 is preferably configured to provide for a sufficient spring force for a minimum dose of drug 31 to be dispensed from the drug delivery device 200 when the piston rod 6 is in the second position. Due to the threaded interaction of the dose member 2 and the drive member 1, the drive member 1 is moved distally with respect to the piston rod 6 when the dose member 2 rotates. The piston rod 6 comprises a piston rod feature 17 and the drive member 1 comprises a drive feature 15 (cf. FIG. 2). By an engagement of the drive feature 15 with the piston rod feature 17, a distal movement of the drive member 1 may be transferred to the piston rod 6 such that the piston rod 6 is moved distally with respect to the housing 24. Consequently, the plunger 19 is moved distally inside the cartridge 14 in order to dispense drug 31 from the drug delivery device 200. The drug delivery device 200 further comprises a coupling element 16 which is configured such that the piston rod 6 is prevented from being moved in the proximal direction. When the dose button 5 is released either during or after a dispensing operation, the releasable clutch mechanism is reengaged and the drive member 1 is moved back from the second into the first position thus switching the drive mechanism back into the setting mode of operation. The drug delivery device 200 may be an injector-type device comprising a needle or a needle assembly (not explicitly indicated) which may be provided at the distal end 25. Furthermore, the drug delivery device 200 may comprise a cap (not explicitly indicated) to cover the distal end 25. The dose button 5 may need to be rotated six times during setting of a dose. This may correspond to a set dose of 120 units of drug 31 to be dispensed.

In the following, a priming mechanism is described by which the drug delivery device 200 may be switched from an initial state to a primed state. In the initial state, the drug delivery device 200 is preferably in an as-fabricated or as-assembled state, wherein the dose button 5 has not yet been actuated or pressed. Then, the cartridge 14 preferably contains an initial amount of drug 31. The rod displacement feature 30 is axially movable between a first position and a second position. In the initial state, the piston rod 6 and the rod displacement feature 30 are arranged such that some or all of the movement of the rod displacement feature 30 from the first position to the second position is transferred to the piston rod 6 such that the piston rod 6 is moved with respect to the cartridge 14. In the primed state, axial movement of the rod displacement feature 30 from the first position to the second position is not transferred to the piston rod 6. Particularly, when the dose button 5 is in the initial state, a proximal face 47 of the piston rod 6 and a distal face 46 of the rod displacement feature 30 preferably abut (cf. also FIG. 6). Alternatively, the distance between the proximal face 47 of the piston rod 6 and a distal face 46 of the rod displacement feature 30 is at least smaller than the distance between the first and the second position. When, in the initial state, the dose button 5 is pressed by the user for the first time, the rod displacement member 30 is moved axially, thereby moving or advancing the piston rod 6 distally with respect to the cartridge 14. Expediently, a needle is provided which in turn provides fluid communication between the interior of the cartridge 14 and the outside. The dose button 5 is moved until the rod displacement feature 30 is arranged in the second position, whereby an initial static friction force between the plunger 19 and the cartridge 14 is overcome. Thereby, the drug delivery device 200 is primed. The priming operation may additionally comprise the removal of clearances and/or tolerances and the application of compression or tension to further device components such that the device is prepared for an operation with no or only a minimum play between elements of the drive mechanism. The use of force transferred from the rod displacement member 30 to the piston rod 6 may help the drive mechanism to overcome initial static friction forces, particularly between the plunger 19 and the cartridge 14.

In the primed state, the driving force is preferably sufficient to move or advance the piston 19 distally with respect to the cartridge 14. As an advantage, the drive spring 13 may be designed smaller and more efficient with respect to costs and space requirements. In the initial state, the distance between the piston rod 6 and the rod displacement feature 30 is preferably greater than a manufacturing tolerance of the cartridge 14 and/or the piston rod 14. Thereby, it is assured, that the direct distal movement of the rod displacement feature—which is effected manually by the user—is effectively transferred to the piston rod 6. Preferably, the plunger 19 and the cartridge 14 are configured such that the initial static friction force takes values between 10 N and 20 N. Preferably, the mentioned drive mechanism is configured such that the driving force takes values between 3 N and 10 N.

The distance by which the dose button 5 is depressed may be 3 to 4 mm. The rod displacement feature 30 may be moved axially between the first and the second position. The distance the rod displacement feature 30 is moved axially may be 2 mm. Additionally, there may be a play or clearance of distance B of 1 to 2 mm between the dose button 5 and the displacement member 3 (cf. FIG. 6). To this effect, a further biasing member (not explicitly indicated) may be provisioned which tends to separate the mentioned components accordingly.

An advantage of the described priming functionality pertains to the effect that once the drug delivery device 200 is primed, the user may repeat the actuation or depressing of the dose button 5 if he is not sure about the state of the device. By means of the force necessary to depress the dose button 5, the user will immediately realize whether the device has already been primed or not. Thereby, it is contributed to a simple and safe operation of the drug delivery device 200. The presented drug delivery device 200 provides the advantages of, for instance, a comfortable, user-friendly shape, a low injection force owing to the priming mechanism, semi-automatic injection and the possibility to assemble the drug delivery device in an easy way. Moreover, the drug delivery device may be easily distinguished from other devices due to its characteristic shape. That is to say, the shape of the drug delivery device may deviate slightly from a cylinder-like shape which is usual for similar drug delivery devices. To this effect, it may be easier to hold it in the palm of the user's hand and/or to operate the drug delivery device.

FIG. 2 illustrates a coupling between the piston rod 6 and the coupling element 16 by means of the FIGS. 2a to 2c. Drive feature 15 of the drive member 2 may constitute a plurality of drive features 15 which are shown each with a tilt towards the distal end 25. The depicted situation respectively relates to the setting mode of operation, wherein the drive features 15 are disengaged from the piston rod 6. The coupling element 16 is preferably fixed to or integrally formed with the housing 24. The coupling element 16 comprises three axially spaced coupling features 20 each of which comprises teeth 41. The coupling features 20 are also tilted towards the distal end 25. The piston rod 6 comprises a set of teeth 40 which exhibit the piston rod feature 17. The teeth 40 are configured uniformly and arranged equidistantly. Furthermore, the teeth 40 may be arranged at an inwardly directed, as well as an outwardly directed side surface of the piston rod 6. The side surfaces of the piston rod 6 may be flat or non-flat, such as even. Each of the coupling features 20 is configured to establish a unidirectional coupling with the teeth 40 such that a proximal movement of the piston rod 6 with respect to the housing 24 is prevented. In FIG. 2a, only the coupling feature 20b in the middle establishes said unidirectional coupling, as proximal end faces of the teeth 40 abut distal end faces of teeth 41 of that coupling element while this is not the case for the other coupling features 20b, c. The distance between the proximal end faces of two adjacent teeth 40 of the piston rod 6 is indicated by A. The axial distance between the coupling features 20 is chosen such that the distance D between a distal end face of a tooth 41 of the coupling feature 20c which does not establish the unidirectional coupling and a proximal end face of a tooth 40 of the piston rod 6 feature is smaller than the distance A. Preferably, the distance D corresponds to a minimum amount of a drug 31 to be dispensed from the delivery device 200. The distance D is preferably defined by those teeth 42 of the piston rod 6 which are arranged proximally next to the respective tooth 41 of the respective coupling element. The distance D corresponding to a minimum amount of a drug 31 to be dispensed is expediently smaller than the distance A. Consequently, the piston rod 6 may be moved distally with respect to the coupling element 16 by a distance smaller than the distance A. In FIG. 2b, as compared to the FIG. 2a, the piston rod 6 has been moved distally (to the left) with respect to the coupling element 16 by the distance D. Thereby, the proximal end faces of the teeth 40 of the piston rod 6 have been moved out of abutment with the distal end faces of the teeth 41 of the middle coupling feature 20b and proximal end faces of the teeth 40 abut distal end faces of the left coupling feature 20a such that only this coupling feature establishes unidirectional coupling to the piston rod 6. In FIG. 2c, the piston rod 6 has been moved further axially by the distance D′, as compared to FIG. 2b. As a consequence, only the right coupling feature 20c forms the mentioned unidirectional coupling to the piston rod 6. The distance D′ may relate to the distance D.

The drive features 15 of the drive member 1 are axially spaced, wherein each drive feature 15 comprises teeth 42 being configured to be engagable with the teeth 40 of the piston rod 6, wherein the drive features 15 and the teeth 42 are configured such that when the drive member 2 is in the second position, a distal end faces of a teeth 42 of one of the drive features 15 abut proximal end faces of teeth 40 of the piston rod 6 and distal end faces of a teeth 42 of another drive feature 15 are spaced preferably by the distance D from proximal end faces of a teeth 40 of the piston 6.

The functionality which is described herein by means of three teeth 41 may also function with coupling features and drive features which only comprise one tooth each.

However, the provision of a plurality of teeth adds greater strength and some redundancy in the case of failure.

FIG. 3a shows a partial perspective view of inner components of the drug delivery device 200 in the setting mode of operation. The displacement member 3 comprises a clutch feature 33 which is, in the depicted situation, engaged to the pinion 11 of the dose member 2. In this situation, the releasable clutch mechanism is engaged. The pinion comprises teeth 48. The clutch feature 33 is a tooth-like clutch feature which is engaged or splined to the teeth 48 (cf. dashed circle). Although the dose member 2 may be selectively rotationally locked with respect to the housing 24 against the spring force of the drive spring 13, it may still be rotated, e.g. by a rotation of the dose button 5 with respect to the housing 24 which is performed by the user. During a setting operation, particularly during a clockwise rotation (cf. arrow 29 in FIG. 1) of the dose member 2 with respect to the displacement member 3, said clutch interaction between the clutch feature 33 and the pinion 11 may be overcome by the user. The torque required to set a dose may be 13.4 mNm for a set dose of zero and 25.4 mNm for a set dose of 120 units. The torque required to decrease or cancel a dose may be 16.8 mNm for a minimum dose and 4.8 mNm for the maximum settable dose. Expediently, said torques are larger than the torque which is applied to the dose member 2 by the drive spring 13. FIG. 3b shows the situation in the dispensing mode of operation, wherein the clutch feature 33 has been disengaged from the teeth 48 of the pinion 11, by movement of the dose button 5 with respect to the housing 24. As compared to FIG. 3a, also the displacement member 3 has been moved distally with respect to the dose member 2. Consequently, in FIG. 3b, the dose member 2 is free to rotate with respect to the displacement member 3. In this situation, it is enabled that the drive spring 13 drives the dose member 2 in the second direction or counter clockwise (cf. arrow 32 in FIG. 1) by rotation.

FIG. 4 shows parts of components of the drug delivery device 200 near the proximal end 26. In the setting mode of operation, the dose button 5 is rotationally locked to the dose member 2 by a dose member spline 35 which is retained in a recess 34 of the dose button 5 such that the dose member 2 is rotated along with the dose button 5. In the dispensing mode of operation, the dose member 2 is free to rotate with respect to the dose button 5. To this effect, the dose member spline 35 and the recesses 34 are configured such that when the dose button 5 is moved distally with respect to the dose member 2, e.g. during a dispensing operation, the dose member spline 35 is disengaged from the corresponding recess 34 and the dose member 2 is free to rotate with respect to the dose button 5. When the dose button 5 is released again, e.g. during or after a dispensing operation, the dose member spline 35 is reengaged to one of the recesses 34, e.g. to that recess which is arranged closest to the dose member spline 35. This is due to the effect of the spring element 4 which biases the dose member 2 towards the distal end 25.

FIG. 5 shows a perspective view of components of the drug delivery device 200 illustrating, e.g. the function of a last dose mechanism of the drug delivery device 200. A last dose ratchet 37 is provisioned to hinder, e.g. a user to set a further dose when the maximum settable dose is already reached or set. When a dose of a drug 31 is set, the drive member 1 moves away from the distal end 25. When a dose is dispensed, the piston rod 6 moves distally with respect to the housing 24 together with the drive member 1. The drive member 1 comprises a drive member arm 39. The last dose ratchet 37 may be borne or retained by the pins 43 which may interact with a further component of the drug delivery device 200 or the housing 24 such that the last dose ratchet 37 is rotated around an axis defined by the pins 43. The dose member 2 comprises a dose member ratchet 38 further comprising teeth 44 which are disposed on a circumferential face or another face. When a large or a maximum number of units has already been dispensed from the drug delivery device 200 and/or when a large or a maximum dose is set, the drive member arm 39 engages a ramp 36 near the proximal end 26. Thereby, the drive member arm 39 is moved radially outwards, thus rotating the last dose ratchet 37 around the axis which extends through the pins 43. Due to the rotation of the last dose ratchet 37, an end portion 45 of the last dose ratchet 37 engages the teeth 44 of the dose member ratchet 38 such that the dose member 2 is prevented from rotation in the first direction 29, i.e. the direction in which the set dose is increased. In the setting mode of operation, the dose member 2 may then be moved in the second direction (cf. arrow 32 in FIG. 1) in order to decrease or cancel the dose. Thereby, the drive member 1 is moved distally with respect to the piston rod 6. When the maximum dose is set, the dose member 2 is thus prevented from being rotated in the first direction 29 to set an additional dose of drug 31.

FIG. 6 shows a partial side view of inner components of the drug delivery device 200. In the depicted situation, the drug delivery device 200 is in an initial state and the rod displacement feature 30 abuts or is closely arranged to the piston rod 6. When the dose button 5 is then moved or depressed in the distal direction by a distance B which may correspond to 1 mm, the dose button abuts the displacement member 3. Upon further distal movement of the dose button 5, the displacement member 3 and also the piston rod 6 is moved distally against the resilience of the spring element 4. The indication assembly 100 is shown in FIG. 6 in greater detail. The drug delivery device 200 may comprise a window 21 which may be comprised by the housing 24. The dose member 2 comprises a first pinion 9. The pinion 11 exhibits a second pinion, as mentioned above. The first indication member 110 further comprises a first corresponding pinion 115 which is engaged or engagable to the first pinion 9. The first pinion 9 comprises a protrusion 49. The second indication member 120 further comprises a second corresponding pinion 125 which is engaged to the second pinion 11. The first and the second indication member 110, 120 comprise indication numbers or symbols which, e.g. to indicate the size of a set dose. The first and the second indication members 110, 120 are mechanically decoupled from one another. The dose member 2 may be suitable to drive the first and the second indication member 110, 120, particularly to rotate said components via the first and the second pinion 9, 11 with respect to the housing 24. The first indication member 110 may be incrementally rotatable such that when the dose member 2 is rotated by one revolution, the first indication member 110 is rotated by one increment due to an engagement of the protrusion 49 with the first corresponding pinion 115. The locking member 130 may be rotationally locked with respect to the housing 24 of the drug delivery device 200. Thereby, the housing 24 may be and/or act as a locking member guide. The first and the second indication members 110, 120 are rotatable with respect to the locking member 130. The axis of rotation may be an axis parallel to the longitudinal axis x of the drug delivery device 200. The locking member 130 may be axially movable respect to the housing 24. In FIG. 6, a rotation of the dose member 2 in the first direction would lead to a rotation of the second indication member 120 in the second direction such that the number 1 would be indicated through the window 21. In FIG. 7 the counting direction of the depicted numbers of the first and second indication member 110, 120 is reversed for the sake of clarity.

By means of FIG. 7 the indication assembly 100 is described schematically and in greater detail. The indication assembly 100 may contribute to a display mechanism of the drug delivery device 200 which allows, e.g. for an indication and/or counting of set doses of the drug delivery device 200. The first indication member 110 comprises a first indication feature 111 and the second indication member 120 comprises a second indication feature 122 which may be a recess. The first indication feature 111 comprises a guiding surface 112 and a stop face 113. The locking member 130 comprises a first locking member feature 131 and a second locking member 132. The first indication member 110 further comprises a cut-out 114. The first and the second locking member feature 131, 132 are arranged on opposite sides of the locking member 130.The first and the second indication member 110, 120 are at least partially arranged axially between the first and the second locking member feature 131, 132.

In FIG. 7a, a dose of zero units is indicated. The locking member 130 is arranged in the cut-out 114. Via the cut-out the first indication member 110 is rotationally locked in the second direction with respect to the locking member 130. The second indication member 120 further comprises a second interaction feature or stop 121. The locking member 130 is arranged at an axial position, wherein it abuts the stop 121, thereby simultaneously locking the second indication member 120 rotationally in the second direction with respect to the locking member 130. This situation may correspond to an initial position of the locking member 130, wherein the set dose is zero. The first and the second indication member 110, 120 may comprise cylindrical indication surfaces. The first and the second locking member features 131, 132 comprise one or more even surfaces which are arranged obliquely with respect to the axis of rotation of the first or the second indication member 110, 120. The first and the second indication member 110, 120 may rotate around a common axis, preferably around the longitudinal axis x. When a dose of the drug delivery device 200 is set, the dose member 2 is rotated in the first direction via the dose button 5 such that this set dose may be indicated through the window 21. Such rotation may relate to a rotation of the first and the second indication member 110, 120 in a first rotational direction with respect to the locking member 130. A rotation of the dose member 2 in the second direction relates, accordingly, to a rotation of the first and the second indication member 110, 120 in a second rotational direction with respect to the locking member 130. When a dose of the drug delivery device 200 is set, the second pinion 11, drives the second corresponding pinion 125 of the second indication member 120 such that the set dose, i.e. the dose indicated by the indication assembly 100 increases. Thus, when, in FIG. 7, the indicated dose (which may correspond to the number in the middle) is increased, the indication assembly 100 and the dose member 2 are configured such that, when the second indication member 120 is rotated for one revolution, the first indication member 110 is rotated by an angle corresponding to one digit such that “1” is indicated instead of “0”. Thereby, the locking member 130 is moved axially and out of the cut-out 114 of the first indication member 110. Now the locking member 130 is in a non-locking position, as, for example, shown in FIG. 7c by means of the indicated dose of 51 units. The locking member 130 is biased towards said non-locking position by a biasing member (not explicitly indicated) with respect to the first and the second indication members 110, 120. Preferably, the locking member is biased towards the initial position of the locking member 130. When the maximum dose of, for example 120 units, is set (cf. FIG. 7b), which may correspond to a predetermined angle of the first and/or the second indication member 110, 120 with respect to the locking member 130, the first locking member feature 131 interacts with the first interaction feature 111 such that the locking member 130 is displaced into a locking position (cf. FIG. 7b) with respect to the indication member 130, thereby rotationally locking the first and the second indication member 110, 120 with respect to the locking member 130 in the first direction. The displacement of the locking member 130 with respect to the first and the second indication member 110, 120 is an axial displacement, e.g. along the longitudinal axis x of the drug delivery device 200 (cf. FIG. 1). In the second direction, the first and the second indication member 110, 120 are still rotatable with respect to the locking member 130. The rotation in the second direction may relate to an operation of the drug delivery device 200, wherein a dose is decreased or cancelled. The axial displacement of the locking member 130 is guided by the guiding surface 112 such that the second locking member feature 132 is axially displaced into the second indication feature 122. In FIG. 7b, the locking member 130 abuts the stop face 113. Now, the first and the second indication member 110, 120 are rotationally locked in the first direction with respect to the locking member 130. This situation may relate to an indication of the maximum settable dose (cf. “120” in FIG. 7b).

As an advantage of the presented indication assembly, a display or indication mechanism for the drug delivery device can be provided which functions with a low number mutually interacting components and thus the counting assembly can be embodied robust and safe.

An application of the presented indication assembly is not restricted to drug delivery device, but merely illustrated exemplarily by that means.

The end of a dose delivery action may be indicated by a feature (not explicitly indicated) which provides an audible feedback when further components of the drug delivery device move relative to one another.

The drug delivery device may additionally comprise components which are not explicitly indicated and/or the function of which is not described herein. For instance, the presented device may comprise a mechanism which decouples or ratchets the dose button relative to the dose member if an excessive torque is applied to the dose button by the user in the setting mode of operation.

Although this is not explicitly described herein, the actuation member and the displacement member may be embodied in a single component and the whole configuration of the drive mechanism and/or the device may be adjusted accordingly.

A further variant of the design may allow the user to decouple the coupling feature from the piston rod feature such that the piston rod can be moved axially, e.g. proximally, with respect to the housing. This is particularly advantageous when the drug delivery device is configured reusable such that the piston rod can be reset and the cartridge can be changed.

The drive mechanism may further comprise a clutch element which is configured to receive motion of the actuation member when the user attempts to set a dose greater than the maximum settable dose. This may be embodied by means of a torque limiter comprising, e.g. protrusions, recesses and/or resilient elements preventing destruction or damage of components of the drive mechanism and/or the drug delivery device when the user attempts to set a dose greater than the maximum settable dose.

The term “drug” or “substance”, as used herein, preferably means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy, wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivatives are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoryl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH 2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36[Asp28] Exendin-4(1-39),

des Pro36[IsoAsp28] Exendin-4(1-39),

des Pro36[Met(O)14, Asp28] Exendin-4(1-39),

des Pro36[Met(O)14, IsoAsp28] Exendin-4(1-39),

des Pro36[Trp(O2)25, Asp28] Exendin-4(1-39),

des Pro36[Trp(O2)25, IsoAsp28] Exendin-4(1-39),

des Pro36[Met(O)14 Trp(O2)25, Asp28] Exendin-4(1-39),

des Pro36[Met(O)14 Trp(O2)25, IsoAsp28] Exendin-4(1-39); or

des Pro36[Asp28] Exendin-4(1-39),

des Pro36[IsoAsp28] Exendin-4(1-39),

des Pro36[Met(O)14, Asp28] Exendin-4(1-39),

des Pro36[Met(O)14, IsoAsp28] Exendin-4(1-39),

des Pro36[Trp(O2)25, Asp28] Exendin-4(1-39),

des Pro36[Trp(O2)25, IsoAsp28] Exendin-4(1-39),

des Pro36[Met(O)14 Trp(O2)25, Asp28] Exendin-4(1-39),

des Pro36[Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36[Asp28] Exendin-4(1-39)-Lys6-NH2,

des Asp28 Pro36, Pro37, Pro38Exendin-4(1-39)-NH2,

H-(Lys)6-des Pro36, Pro38[Asp28] Exendin-4(1-39)-NH2,

H-Asn-(Glu)5des Pro36, Pro37, Pro38[Asp28] Exendin-4(1-39)-NH2,

des Pro36, Pro37, Pro38[Asp28] Exendin-4(1-39)-(Lys)6-NH2,

H-(Lys)6-des Pro36, Pro37, Pro38[Asp28] Exendin-4(1-39)-(Lys)6-NH2,

H-Asn-(Glu)5-des Pro36, Pro37, Pro38[Asp28] Exendin-4(1-39)-(Lys)6-NH2,

H-(Lys)6-des Pro36[Trp(O2)25, Asp28] Exendin-4(1-39)-Lys6-NH2,

H-des Asp28 Pro36, Pro37, Pro38[Trp(O2)25] Exendin-4(1-39)-NH2,

H-(Lys)6-des Pro36, Pro37, Pro38[Trp(O2)25, Asp28] Exendin-4(1-39)-NH2,

H-Asn-(Glu)5-des Pro36, Pro37, Pro38[Trp(O2)25, Asp28] Exendin-4(1-39)-NH2,

des Pro36, Pro37, Pro38[Trp(O2)25, Asp28] Exendin-4(1-39)-(Lys)6-NH2,

H-(Lys)6-des Pro36, Pro37, Pro38[Trp(O2)25, Asp28] Exendin-4(1-39)-(Lys)6-NH2,

H-Asn-(Glu)5-des Pro36, Pro37, Pro38[Trp(O2)25, Asp28] Exendin-4(1-39)-(Lys)6-NH2,

H-(Lys)6-des Pro36[Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38[Met(O)14, Asp28] Exendin-4(1-39)-NH2,

H-Asn-(Glu)5-des Pro36, Pro37, Pro38[Met(O)14, Asp28] Exendin-4(1-39)-NH2,

des Pro36, Pro37, Pro38[Met(O)14, Asp28] Exendin-4(1-39)-(Lys)6-NH2,

H-(Lys)6-des Pro36, Pro37, Pro38[Met(O)14, Asp28] Exendin-4(1-39)-(Lys)6-NH2,

H-Asn-(Glu)5 des Pro36, Pro37, Pro38[Met(O)14, Asp28] Exendin-4(1-39)-(Lys)6-NH2,

H-Lys6-des Pro36[Met(O)14, Trp(O2)25, Asp28] Exendin-4(1-39)-Lys6-NH2,

H-des Asp28 Pro36, Pro37, Pro38[Met(O)14, Trp(O2)25] Exendin-4(1-39)-NH2,

H-(Lys)6-des Pro36, Pro37, Pro38[Met(O)14, Asp28] Exendin-4(1-39)-NH2,

H-Asn-(Glu)5-des Pro36, Pro37, Pro38[Met(O)14, Trp(O2)25, Asp28] Exendin-4(1-39)-NH2,

des Pro36, Pro37, Pro38[Met(O)14, Trp(O2)25, Asp28] Exendin-4(1-39)-(Lys)6-NH2,

H-(Lys)6-des Pro36, Pro37, Pro38[Met(O)14, Trp(O2)25, Asp28] Exendin-4(S1-39)-(Lys)6-NH2,

H-Asn-(Glu)5-des Pro36, Pro37, Pro38[Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropin (Follitropin, Lutropin, Choriongonadotropin, Menotropin), Somatropin (Somatropin), Desmopressin, Terlipressin, Gonadorelin, Triptorelin, Leuprorelin, Buserelin, Nafarelin, Goserelin.

A polysaccharide is for example a glucosaminoglycan, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and Υ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, αand δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by μ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystallizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts.

Acid addition salts are e.g. HCI or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The scope of protection of the invention is not limited to the examples given hereinabove. The invention is embodied in each novel characteristic and each combination of characteristics, which particularly includes every combination of any features which are stated in the claims, even if this feature or this combination of features is not explicitly stated in the claims or in the examples.

### REFERENCE NUMERALS

1 Drive member

2 Dose member

3 Displacement member

4 Spring element

5 Dose button

6 Piston rod

9 First pinion

11 Second pinion

13 Drive spring

14 Cartridge

15 Drive feature

16 Coupling element

17 Piston rod feature

19 Plunger

20 Coupling feature

21 Window

22 Outer thread

23 Guide feature

24 Housing

25 Distal end

26 Proximal end

27 Guidance

28 Bore

29 First direction

30 Rod displacement feature

31 Drug

32 Second direction

33 Clutch feature

34 Recess (dose button)

35 Dose member spline

36 Ramp

37 Last dose ratchet

38 Dose member ratchet

39 Drive member arm

40 Teeth (piston rod)

41 Teeth (coupling element)

42 Teeth (drive feature)

43 Pin

44 Teeth (dose member ratchet)

45 End portion

46 Distal face

47 Proximal face

48 Teeth (second pinion)

49 Protrusion

50 Drive member displacement member

100 Indication assembly

110 First indication member

111 First interaction feature

112 Guiding surface

113 Stop face

114 Cut-out

115 First corresponding pinion

120 Second indication member

121 Second interaction feature

122 Second indication feature/recess

125 Second corresponding pinion

130 Locking member

131 First locking member feature

132 Second locking member feature

200 Drug delivery device

x Longitudinal axis

A,B,D Distance

1. Indication assembly (100) comprising:
an indication member (110) comprising an interaction feature (111), the indication member (110) being rotatable and
a locking member (130) being movable with respect to the indication member (110) between a locking position and a non-locking position, the locking member (130) comprising a locking member feature (131), wherein
the indication member (110) is rotatable relative to the locking member (130) around an axis (x), and wherein
the indication assembly (100) is configured such that:
when the locking member (130) is in the non-locking position, the indication member (110) is rotatable by a predetermined angle with respect to the locking member (130) in a first rotational direction and
when the locking member (130) is in the locking position, the indication member (110) is rotationally locked with respect to the locking member (130) in the first rotational direction and
when the locking member (130) is in the non-locking position and the indication member (110) is rotated by the predetermined angle with respect to the locking member (130), the locking member feature (131) interacts with the interaction feature (111) such that the locking member (130) is displaced into the locking position with respect to the indication member (110), thereby rotationally locking the indication member (110) with respect to the locking member (130) in the first rotational direction.

2. Indication assembly (100) according to claim 1, wherein the indication assembly (100) is configured such that the movement of the locking member (110) between the locking position and the non-locking position is or comprises an axial displacement along the axis (x).

3. Indication assembly according to claim 1 or 2, wherein the indication assembly (100) is configured such that, when the locking member (130) is arranged in the locking position, the indication member (110) is rotatable in a second rotational direction with respect to the locking member (130), wherein the second rotational direction is opposite to the first rotational direction.

4. Indication assembly (100) according to at least one of the claims 1 to 3, wherein the interaction feature (111) comprises a stop face (113) and a guiding surface (112).

5. Indication assembly (100) according to at least one of the previous claims, wherein the interaction feature (111) comprises a stop face (113) and a guiding surface (112), the stop face (113) having a surface normal which is perpendicular to the axis (x), wherein the guiding surface (112) is arranged adjacent to the stop face (113) and the guiding surface (112) is arranged to guide the locking member feature (131) against the stop face (113) such that the indication member (110) is rotationally locked with respect to the locking member (130) in the first rotational direction by abutment of the locking member feature and the stop face (113).

6. Indication assembly (100) according to at least one of the previous claims, further comprising a locking member guide, wherein the locking member (130) is axially guided by the locking member guide.

7. Indication assembly (100) according to at least one of the previous claims, wherein the indication member (110) is a first indication member (110) and the interaction feature (111) is a first interaction feature (111), wherein the indication assembly (100) comprises a second indication member (120) and the second indication member (120) comprises a second interaction feature (121) and the first and the second indication member (110, 120) cooperate to define information indicated by the indication assembly (100), and wherein the locking member feature (131) is a first locking member feature (131) and the locking member (130) comprises a second locking member feature (132) which is arranged and configured such that it rotationally locks the second indication member (120) in the first rotational direction with respect to the locking member (130) when the first and/or the second indication member (110, 120) are rotated by the predetermined angle in the first rotational direction with respect to the locking member (130).

8. Indication assembly (100) according to claim 7, wherein the guiding surface (112) is arranged obliquely with respect to an axis (x) of rotation and the second interaction feature (121) comprises a recess and the guiding surface (112) and the recess are configured such that when the first and/or the second indication member (110, 120) are rotated by the predetermined angle in the first rotational direction with respect to the locking member (130), the locking member (130) is guided by the guiding surface (112) such that the second locking member feature (132) is displaced into the recess.

9. Indication assembly (100) according to claim 7 or 8, wherein the first and the second indication member (110, 120) are configured such that in a first position of the first and the second indication member (110, 120), the first and the second indication member (110, 120) are rotationally locked by and with respect to the locking member in the second rotational direction.

10. Indication assembly (100) according to one of the previous claims, wherein the locking member (130) is biased towards the non-locking position by a biasing member.

11. System comprising the indication assembly (100) according to at least one of the claims 7 to 10, wherein the first and the second locking member feature (131, 132) are arranged at or near opposite ends of the locking member (130) and wherein the first and the second indication member (110, 120) are at least partially arranged axially between the first and the second locking member feature (131, 132).

12. System according to claim 11 comprising a driver (2) which is coupled to the first and the second indication member (110, 120).

13. System according to claim 12, wherein the driver (2) comprises a first pinion (9) and a second pinion (11) and the first indication member (110) comprises a first corresponding pinion (115) being coupled to the first pinion (11) and the second indication member (120) comprises a second corresponding pinion (125) being coupled to the second pinion (11), wherein the driver (2) is suitable to drive the first and the second indication member (110, 120) via the first and the second corresponding pinion (115, 125).

14. System according to claim 13, wherein the first indication member (110) is incrementally rotatable and the coupling between the driver (2) and the first and second indication member (110, 120) is configured such that when the second indication member (120) is rotated by one revolution, the first indication member (110) is rotated by one increment.

15. Drug delivery device (200) comprising the indication assembly (100) according to at least one of claims 1 to 10 or the system according to at least one of claims 11 to 14 and a housing (24) having a proximal and a distal end (25, 26), wherein the drug delivery device (200) is a variable dose device, wherein the size of a dose of drug (31) to be dispensed by the device can be set by a user between a minimum dose and a maximum dose, the indication assembly (100) is adapted to indicate the size of the currently set dose and, when the maximum dose is set the locking member (130) is in the locking position.

16. Use of the indication assembly (100) according to at least one of claims 1 to 10 or of the system according to at least one of the claims 11 to 14 as a display mechanism for a drug delivery device (200), preferably as a dose display mechanism, e.g. a dose display mechanism which simultaneously provides a maximum settable dose stop which defines the maximum dose of drug (31) which can be set to be dispensed by the drug delivery device (200) in a single dispensing action.
